(19) United States
(12) Patent Application Publication
(10) Pub. No.: US 2022/0072087 A1
(43) Pub. Date: Mar. 10, 2022

(54) SPECIFIC COMBINATION THERAPY FOR TREATMENT OF PANCREATIC CANCER

(71) Applicants: BioLineRx Ltd., ModiIn (IL); Biokine Therapeutics Ltd., Nes Ziona (IL)

(72) Inventors: Gideon STEIN, Rosh HaAyin (IL); Amnon PELED, Tel-Aviv (IL)

(73) Assignees: BioLineRx Ltd., ModiIn (IL); Biokine Therapeutics Ltd., Nes Ziona (IL)

(21) Appl. No.: 17/422,786

(22) PCT Filed: Oct. 17, 2019
(86) PCT No.: PCT/IL2019/051127

§ 371 Date: Jul. 14, 2021

Related U.S. Application Data
(60) Provisional application No. 62/793,414, filed on Jan. 17, 2019.

(51) Int. Cl. A61K 38/10; A61K 31/4745; A61K 31/513; A61K 31/519; A61K 45/06

(52) U.S. Cl. CPC A61K 38/10; A61K 31/4745; A61K 45/06; A61K 31/519; A61K 31/513

(57) ABSTRACT

A method of treating metastatic pancreatic adenocarcinoma in a subject in need thereof is provided. The method comprises administering to the subject a therapeutically effective amount of a peptide set forth in SEQ ID NO: 1 and a chemotherapy comprising irinotecan, fluorouracil (5-FU) and leucovorin (LV), thereby treating the metastatic pancreatic adenocarcinoma, wherein the subject is not subjected to treatment with an anti-PD-1.

### RELATED APPLICATION

This application claims the benefit of priority from U.S. Provisional Patent Application No. 62/793,414, filed Jan. 17, 2019, which is hereby incorporated by reference in its entirety.

### SEQUENCE LISTING STATEMENT

The ASCII file, entitled 78930 Sequence Listing.txt, created on 8 Sep. 2019, comprising 903 bytes, submitted concurrently with the filing of this application is incorporated herein by reference.

### FIELD AND BACKGROUND OF THE INVENTION

The present invention, in some embodiments thereof, relates to the treatment of pancreatic cancer using a specific drug combination.

Pancreatic cancer is chemotherapy-resistant, with an extremely poor prognosis. It is the fourth leading cause of cancer death in the United States; the 5-year survival rate is 6%. The incidence of pancreatic cancer has increased during the past several decades and in 2014, an estimated 46,420 patients were diagnosed with pancreatic cancer and 39,590 died. Pancreatic cancer is projected to surpass liver, breast, prostate, and colorectal cancers to become the second-leading cause of cancer-related death by 2030. These statistics reflect the dire nature of the disease and lack of effective therapies. The location of the tumor results in few early symptoms and is often diagnosed at a late stage as a result. The absence of effective screening tools, and a limited understanding of risk factors, means that patients have advanced or metastatic disease at the time of diagnosis. Given the poor prognosis and the low median survival rates of less than one year for patients with metastatic disease, new treatment options are still needed.

BL-8040 (previously named BKT140, SEQ ID NO: 1) is a highly selective CXCR4 antagonist. The investigational drug is a 14-residue, cyclic, synthetic peptide capped with an aromatic ring that binds to CXCR4 with high affinity (IC₅₀ 0.54-4.5 nM) and inhibits its function[1]. The chemokine CXCL12 (SDF-1-stromal-derived-factor-1) and its receptor, CXCR4, play a pivotal role in the trafficking of hematopoietic cells to the bone marrow (BM)[2]. Animal studies exploring the activity of BL-8040 in various cancer models in mice have shown that in addition to its activity as a mobilizer of hematopoietic cells, BL-8040 exhibits a CXCR4-dependent preferential antitumor effect against malignant cells overexpressing CXCR4[3]. The efficacy of BL-8040 and its analogs for blocking CXCR4 in vitro and in vivo has been documented in numerous preclinical studies, including in vitro and in vivo models for small cell lung carcinoma, breast cancer, malignant melanoma, neuroblastoma and pancreatic cancer. As a CXCR4 antagonist, BL-8040 also affects the trafficking of immune cells to the tumor microenvironment. It was found that administration of BL-8040 induces the mobilization of Natural Killer (NK) cells, T cells and B cells from the BM and lymph nodes into the periphery. Using a syngeneic cancer model in mice it was demonstrated that BL-8040 may eliminate the immunological barrier and allow the accumulation of immune cells within the tumor microenvironment.

Preclinical Studies

The nonclinical development of BL-8040 has encompassed a large number of pharmacodynamic, pharmacokinetic (PK), safety pharmacology, and single and repeated dose toxicity studies.

BL-8040 exhibits CXCR4-dependent selective cytotoxicity toward malignant cells both in vivo and in vitro and induces apoptotic cell death in cancer cells[3-6]. BL-8040 leads to phosphatidylserine externalization, decreased mitochondrial membrane potential, caspase activation, subsequent sub-G1 arrest and DNA double-stranded breaks in leukemic and multiple myeloma cells [3]. These effects were shown to be specific; BL-8040 did not affect the viability of human keratinocytes and normal human hematopoietic cells [3]. This property of direct apoptotic effects on top of the mobilization capacity, distinguishes BL-8040 from other CXCR4 antagonists agents such as Mozobil/Plerixafor [3]. In addition, administration of BL-8040 induces the mobilization of NK cells, T cells and B cells from the BM and lymph nodes into the periphery.

BL-8040 has demonstrated safety and initial clinical efficacy in several Phase I and II studies [Hidalgo M M, Epelbaum R, Semenisty V, Geva R, Golan T, Borazanci E H. Evaluation of pharmacodynamic (PD) biomarkers in patients with metastatic pancreatic cancer treated with BL-8040, a novel CXCR4 antagonist. J Clin Oncol. 2018; 36:88-88. Abstract].

Additional background art includes:

WO2017009843

WO2017009842.

### SUMMARY OF THE INVENTION

According to an aspect of some embodiments of the present invention there is provided a method of treating metastatic pancreatic adenocarcinoma in a subject in need thereof, the method comprising, administering to the subject a therapeutically effective amount of a peptide set forth in SEQ ID NO: 1 and a chemotherapy comprising irinotecan, fluorouracil (5-FU) and leucovorin (LV), thereby treating the metastatic pancreatic adenocarcinoma, wherein the subject is not subjected to treatment with an anti-PD-1.

According to an aspect of some embodiments of the present invention there is provided a therapeutically effective amount of a peptide set forth in SEQ ID NO: 1 and a chemotherapy comprising irinotecan, fluorouracil (5-FU) and leucovorin (LV) for use in treating metastatic pancreatic adenocarcinoma in a subject in need thereof, wherein the subject is not subjected to treatment with an anti-PD-1.

According to some embodiments of the invention, the anti PD-1 is pembrolizumab.

According to some embodiments of the invention, the irinotecan is liposome-encapsulated.

According to some embodiments of the invention, the irinotecan is Onivyde®.

According to some embodiments of the invention, the peptide is administered subcutaneously (SC) or intravenously (IV).

According to some embodiments of the invention, the peptide is administered at a dose of 1.25 mg/kg.

According to some embodiments of the invention, the chemotherapy is administered intravenously.

According to some embodiments of the invention, the irinotecan is administered prior to LV and 5-FU

According to some embodiments of the invention, the irinotecan is administered at a dose of 70 mg/m² as an IV infusion over 90 minutes, followed by LV 400 mg/m² IV over 30 minutes, followed by 5-FU 2400 mg/m² IV over 46 hours, every 2 weeks.

According to some embodiments of the invention, the peptide is administered with the irinotecan.

According to some embodiments of the invention, use of an anti-histamine and optionally analgesics is contemplated.

According to some embodiments of the invention, the subject is post first-line treatment against the metastatic pancreatic adenocarcinoma.

According to some embodiments of the invention, the first-line treatment comprises a gemcitabine-based chemotherapy.

According to some embodiments of the invention, the metastatic pancreatic adenocarcinoma is unresectable.

According to some embodiments of the invention, the metastatic pancreatic cancer is pancreatic ductal adenocarcinoma.

According to some embodiments of the invention, the metastatic pancreatic ductal adenocarcinoma comprises intraductal papillary mucinous neoplasm.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

The present invention, in some embodiments thereof, relates to the treatment of pancreatic cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

According to an aspect of the invention there is provided a method of treating metastatic pancreatic adenocarcinoma in a subject in need thereof, the method comprising, administering to the subject a therapeutically effective amount of a peptide set forth in SEQ ID NO: 1 and a chemotherapy comprising irinotecan, fluorouracil (5-FU) and leucovorin (LV), thereby treating the metastatic pancreatic adenocarcinoma, wherein the subject is not subjected to treatment with an anti-PD-1.

According to an aspect of the invention there is provided a therapeutically effective amount of a peptide set forth in SEQ ID NO: 1 and a chemotherapy comprising irinotecan, fluorouracil (5-FU) and leucovorin (LV) for use in treating metastatic pancreatic adenocarcinoma in a subject in need thereof, wherein the subject is not subjected to treatment with an anti-PD-1.

As used herein “not subjected to treatment with an anti-PD-1” means in the same cycle of treatment. PD-1 can be present in a first line treatment when the present regimen is used as a second or third line treatment.

As used herein “metastatic pancreatic adenocarcinoma” refers to stage IIb to IV of the disease, when the tumor is present out of the pancreas i.e., lymph nodes or other distal locations.

TABLE 1
Stages of pancreatic cancer

| AJCC | Stage | |
|---|---|---|
| Stage | grouping | Stage description* |
| 0 | Tis | The cancer is confined to the top layers of pancreatic duct cells and has not |
| | N0 | invaded deeper tissues. It has not spread outside of the pancreas. These tumors |
| | | are sometimes referred to as carcinoma in situ (Tis). |
| | M0 | It has not spread to nearby lymph nodes (N0) or to distant sites (M0). |
| IA | T1 | The cancer is confined to the pancreas and is no bigger than 2 cm (0.8 inch) |
| | N0 | across (T1). |
| | M0 | It has not spread to nearby lymph nodes (N0) or to distant sites (M0). |
| IB | T2 | The cancer is confined to the pancreas and is larger than 2 cm (0.8 inch) but no |
| | N0 | more than 4 cm (1.6 inches) across (T2). |
| | M0 | It has not spread to nearby lymph nodes (N0) or to distant sites (M0). |
| IIA | T3 | The cancer is confined to the pancreas and is bigger than 4 cm (1.6 inches) |
| | N0 | across (T3). |
| | M0 | It has not spread to nearby lymph nodes (N0) or to distant sites (M0). |
| IIB | T1 | The cancer is confined to the pancreas and is no bigger than 2 cm (0.8 inch) |
| | N1 | across (T1) AND it has spread to no more than 3 nearby lymph nodes (N1). |
| | M0 | It has not spread to distant sites (M0). |
| | T2 | The cancer is confined to the pancreas and is larger than 2 cm (0.8 inch) but no |
| | N1 | more than 4 cm (1.6 inches) across (T2) AND it has spread to no more than 3 |
| | | nearby lymph nodes (N1). |
| | M0 | It has not spread to distant sites (M0). |
| | T3 | The cancer is confined to the pancreas and is bigger than 4 cm (1.6 inches) |
| | N1 | across (T3) AND it has spread to no more than 3 nearby lymph nodes (N1). |
| | M0 | It has not spread to distant sites (M0). |
| III | T1 | The cancer is confined to the pancreas and is no bigger than 2 cm (0.8 inch) |
| | N2 | across (T1) AND it has spread to 4 or more nearby lymph nodes (N2). |
| | M0 | It has not spread to distant sites (M0). |
| | OR | |
| | T2 | The cancer is confined to the pancreas and is larger than 2 cm (0.8 inch) but no |
| | N2 | more than 4 cm (1.6 inches) across (T2) AND it has spread to 4 or more nearby |
| | | lymph nodes (N2). |
| | M0 | It has not spread to distant sites (M0). |
| | OR | |
| | T3 | The cancer is confined to the pancreas and is bigger than 4 cm (1.6 inches) |
| | N2 | across (T3) AND it has spread to 4 or more nearby lymph nodes (N2). |
| | M0 | It has not spread to distant sites (M0). |
| | OR | |
| | T4 | The cancer is growing outside the pancreas and into nearby major blood |
| | Any N | vessels (T4). The cancer may or may not have spread to nearby lymph nodes |
| | | (Any N). |
| | M0 | It has not spread to distant sites (M0). |
| IV | Any T | The cancer has spread to distant sites such as the liver, peritoneum (the lining |
| | Any N | of the abdominal cavity), lungs or bones (M1). It can be any size (Any T) and |
| | M1 | might or might not have spread to nearby lymph nodes (Any N). |

Adapted from - www(dot)cancer(dot)org/cancer/pancreatic-cancer/detection-diagnosis-staging/staging(dot)html

According to a specific embodiment, the metastatic pancreatic adenocarcinoma is pancreatic ductal adenocarcinoma.

As used herein “pancreatic ductal adenocarcinoma” (PDAC) is a type of exocrine pancreatic cancer. It develops from cells lining small tubes in the pancreas called ducts (duct cells in the diagram above). These carry the digestive juices, which contain enzymes, into the main pancreatic duct and then on into the duodenum (first part of the small intestine). PDAC can grow anywhere in the pancreas, although it is most often found in the head of the pancreas.

According to a specific embodiment, the PDAC comprises intraductal papillary mucinous neoplasm.

In some embodiments, the pancreatic cancer is recurrent pancreatic cancer.

In some embodiments, the pancreatic cancer has reoccurred after remission.

In some embodiments, the individual has measurable disease (for example, according to RECIST criteria).

In some embodiments, the individual has one or more metastatic tumors measurable, for example, by CT scan (or MRI).

In some embodiments, the pancreatic cancer is unresectable pancreatic cancer. In some embodiments, the pancreatic cancer is a resectable pancreatic cancer.

In some embodiments, the pancreatic cancer is borderline resectable.

In some embodiments, the primary location of the pancreatic cancer is the head of the pancreas. In some embodiments, the primary location of the pancreatic cancer is the body of the pancreas. In some embodiments, the primary location of the pancreatic cancer is the tail of the pancreas.

As used herein “subject” refers to a human subject diagnosed with metastatic pancreatic adenocarcinoma.

In some embodiments, the subject is a female.

In some embodiments, the individual is a male.

In some embodiments, the individual is under about 65 years old (such as under about any of 60, 55, 50, 45, or 40 years old).

In some embodiments, the subject is at least about 65 years old (such as at least about any of 70, 75, or 80 years old).

According to a specific embodiment, the subject is at least 18 years.

According to a specific embodiment, the subject has not been treated with any previous systemic chemotherapy for the pancreatic adenocarcinoma. Accordingly, treatment is first-line treatment.

According to another embodiment, treatment is first-line treatment against the pancreatic adenocarcinoma.

According to a specific embodiment, the treatment is post first-line treatment against the pancreatic adenocarcinoma.

Thus, methods, regimens, uses, described herein may also be used as a second line or third line therapy after the prior treatment for pancreatic cancer has failed or has substantially failed, or the pancreatic cancer is substantially refractory to the first line therapy. In some embodiments, the individual has received at least one line of therapy (e.g., chemotherapy or immunotherapy) for treating metastatic pancreatic cancer prior to receiving the treatment described herein. In some embodiments, the patient has received 1 line of therapy or 2 lines of therapy (e.g., 1 line of chemotherapy or immunotherapy). Thus, the treatment described herein may be used as a second line therapy. The prior line of therapy described herein may be a prior line of chemotherapy or immunotherapy.

As used herein, the term “treating” includes abrogating, substantially inhibiting, slowing or reversing the progression of a condition, substantially ameliorating clinical symptoms of the metastatic pancreatic adenocarcinoma.

According to a specific embodiment, the subject is diagnosed with metastatic pancreatic adenocarcinoma.

According to a specific embodiment the metastatic pancreatic adenocarcinoma is histologically confirmed (either previously or newly biopsied).

According to a specific embodiment, the subject has a measurable disease (>1 measurable lesion) based on Response Evaluation Criteria In Solid Tumors (RECIST) v1.1.

According to a specific embodiment, tumor lesions situated in a previously irradiated area are considered measurable if progression has been demonstrated in such lesions.

According to a specific embodiment, the subject has histologically confirmed (either previously or newly biopsied) metastatic unresectable pancreatic adenocarcinoma, including with intraductal papillary mucinous neoplasm.

According to a specific embodiment the pancreatic cancer is not acinar cell carcinoma, pancreaticoblastoma, malignant cystic neoplasms, endocrine neoplasms, squamous cell carcinoma. Vater and periampullary duodenal or common bile duct malignancies.

According to a specific embodiment, the subject is not immune-deficient.

According to a specific embodiment, the subject does not have an active autoimmune disease that has required systemic treatment in the 2 years preceding the treatment (i.e., with the use of disease-modifying agents, corticosteroids or immunosuppressive drugs).

According to a specific embodiment, the subject does not have a history of (non-infectious) pneumonitis that required steroids or current pneumonitis.

According to a specific embodiment, the subject does not have a history of interstitial lung disease.

As used herein “a peptide set forth in SEQ ID NO: 1” also referred to as BL-8040 (previously named “BKT140”) or “the peptide” is a highly selective CXCR4 antagonist, a novel therapy for the treatment of cancer. The peptide is a 14-residue, cyclic, synthetic peptide capped with an aromatic ring that binds to CXCR4 with high affinity (IC₅₀ 0.54-4.5 nM) and inhibits its function [Tamamura H, Hiramatsu K, Kusano S, Terakubo S, Yamamoto N, Trent J O, et al. Synthesis of potent CXCR4 inhibitors possessing low cytotoxicity and improved biostability based on T140 derivatives. Org Biomol Chem 2003; 1:3656-3662].

According to a specific embodiment, the BL-8040 is manufactured as a white to off-white powder synthetic polypeptide, freely soluble in water and in 0.45% Sodium Chloride (half normal saline). It is manufactured in accordance with current Good Manufacturing Practice (cGMP) by BioConnection B.V. (previously MSD), Kloosterstraat 9, 5349 AB Oss, Netherlands.

According to a specific embodiment the chemotherapy comprises irinotecan, a topoiseomarase inhibitor.

Irinotecan is converted by esterase enzymes into the more active metabolite, SN-38. The chemical name of irinotecan is (S)-4,11-diethyl-3,4,12,14-tetrahydro-4-hydroxy-3,14-dioxo1H-pyrano [4′,4′-:6,7]-indolizino [1,2-b]quinolin-9-yl-[1,4′bipiperidine]-1′-carboxylate. Irinotecan hydrochloride trihydrate is also referred to by the name CPT-11 and by the trade name CAMPTOSAR®.

The topoisomerase inhibitor can be camptothecin conjugated to a biocompatible polymer such as a cyclodextrin or cyclodextrin analog (e.g., sulfonated cyclodextrins). For example, the topoisomerase inhibitor can be a cyclodextrin-containing polymer chemically bound to a camptothecin, irinotecan, SN-38 or other topoisomerase 1 inhibitor compound. A cyclodextrin-camptothecin conjugated topoisomerase 1 inhibitor can be administered at a pharmaceutically acceptable dose including 6, 12, or 18 mg/m2 weekly administration, or 12, 15 or 18 mg/m2 biweekly administration. Examples of camptothecin-cyclodextrin conjugate topoisomerase 1 inhibitors (e.g., the cyclodextrin-containing polymer conjugate with camptothecin designated “CRLX101”), and related intermediates for preparing the same, are disclosed, for example, in Greenwald et al., Bioorg. Med. Chem., 1998, 6, 551-562, as well as United States Patent Application 2010/0247668, United States Patent Application 2011/0160159 and United States Patent Application 2011/0189092.

The topoisomerase inhibitor can also be a liposomal formulation of a topoisomerase inhibitor such as irinotecan, camptothecin or topotecan. Liposomal irinotecan (e.g., MM-398, also called “nal-IRI”) is a highly stabilized liposomal formulation of irinotecan that provides for sustained exposure of irinotecan, and the active metabolite SN-38 in the tumor to a higher proportion of cells during the more sensitive S-phase of the cell cycle. MM-398 is a liposomal irinotecan that has shown promising preclinical and clinical activity in a range of cancer types, and was recently approved in the United States in combination with 5-FU/LV for patients with metastatic adenocarcinoma of the pancreas after disease progression following gemcitabine-based therapy. Compared with free irinotecan, nal-IRI has an extended PK profile with prolonged local tumor exposure of MM-398 and SN-38. Since SN-38 is cleared more quickly from normal tissues than from tumor, it is hypothesized that delayed dosing of veliparib relative to MM-398 will allow for the expected window of maximum irinotecan-induced toxicity to pass in the absence of concurrent veliparib toxicity. However, the tumor levels of SN-38 are predicted to be sustained upon subsequent veliparib dosing, therefore maintaining the ability of both drugs to act on tumor tissue simultaneously and maintain synergy.

One suitable liposomal Top1 inhibitor formulation is liposomal irinotecan available under the brand name ONIVYDE®. (irinotecan liposome injection) (Merrimack Pharmaceuticals, Inc. Cambridge, Mass.), previously designated “MM-398” prior to FDA approval, and liposomal irinotecan products that are bioequivalent to ONIVYDE. The ONIVYDE/MM-398 (irinotecan liposome injection) includes irinotecan as an irinotecan sucrosofate salt encapsulated in liposomes for intravenous use. The drug product liposome is a small unilamellar lipid bilayer vesicle, approximately 110 nm in diameter, which encapsulates an aqueous space which contains irinotecan in a gelated or precipitated state, as the sucrosofate salt. The liposome carriers are composed of 1,2-distearoyl-sn-glycero-3-phosphocholine (DSPC), 6.81 mg/mL; cholesterol, 2.22 mg/mL; and methoxy-terminated polyethylene glycol (MW 2000)-distearoylphosphatidylethanolamine (MPEG-2000-DSPE), 0.12 mg/mL. Each mL also contains 2-[4-(2-hydroxyethyl)piperazin-1-yl]ethanesulfonic acid (HEPES) as a buffer, 4.05 mg/mL; sodium chloride as isotonicity reagent, 8.42 mg/mL, ONIVYDE/MM-398 is believed to include about 80,000 molecules of irinotecan in a gelated or precipitated state as a sucrosofate salt, encapsulated in a liposome of about 100 nm in diameter.

As used herein “leucovorin” refers to folinic acid that is typically administered in combination with Onivyde® and 5-FU.

5-FU is a thymidylate synthase (TS) inhibitor. Interrupting the action of this enzyme blocks synthesis of the pyrimidine thymidine, which is a nucleoside required for DNA replication. Thymidylate synthase methylates deoxyuridine monophosphate (dUMP) to form thymidine monophosphate (dTMP). Administration of 5-FU causes a scarcity in dTMP, so rapidly dividing cancerous cells undergo cell death via thymineless death. Calcium foliate provides an exogenous source of reduced folinates and hence stabilizes the 5-FU-TS complex, hence enhancing 5-FU's cytotoxicity. 5-FU is sold under the brand name Adrucil, among others.

Doses and regimen for chemotherapy treatment of the disease as described herein and according to performance status are well known in the art of oncology.

According to a specific embodiment treatment does not include oxaliplatin.

The peptide and chemotherapy (“agents”) described hereinabove can be administered to the subject per se, or in a pharmaceutical composition being mixed with suitable carriers or excipients. Each of the agents can be formulated in a separate formulation or at least some of them combined to a single formulation.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agents accountable for the biological effect, e.g., SEQ ID NO: 1 and chemotherapy.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, intradermal, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

According to a specific embodiment, the peptide of the invention or the pharmaceutical composition comprising same is administered subcutaneously (SC).

According to a specific embodiment, the peptide of the invention or the pharmaceutical composition comprising same is administered intravenously (IV).

According to a specific embodiment, the chemotherapy or the pharmaceutical composition comprising same is administered intravenously.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

Pharmaceutical compositions suitable for use in context of some embodiments of the invention include compositions wherein the active ingredients are contained in an amount effective to achieve the intended purpose. More specifically, according to specific embodiments, a therapeutically effective amount means an amount of active ingredients effective to prevent, alleviate or ameliorate symptoms of a disorder (i.e., metastatic pancreatic adenocarcinoma) or prolong the survival of the subject being treated.

According to specific embodiments the peptide of the invention or the pharmaceutical composition comprising same is administered in a dose ranging between 0.1 to 10 mg/kg of body weight, between 0.1 to 2 mg/kg of body weight, between 0.1 to 1 mg/kg of body weight, between 0.3 to 10 mg/kg of body weight, between 0.3 to 2.

According to a specific embodiment, BL-8040 is administered at a dose of 1-2 mg/kg body weight.

According to a specific embodiment, the, BL-8040 is administered at a dose of 1.25-1.5 mg/kg body weight.

According to a specific embodiment, the BL-8040 is administered at a dose of 1.25 mg/kg body weight.

According to a specific embodiment, the BL-8040 is administered subcutaneously (SC).

According to a specific embodiment, the chemotherapy is administered intravenously.

According to a specific embodiment, the peptide is administered with the chemotherapy (e.g., on the same day) or at least one of the chemotherapies. According to a specific embodiment, the peptide is administered in the same formulation as at least one of the chemotherapies, or in separate formulations.

According to a specific embodiment, the chemotherapy is administered according to the tolerated dose which is known in the field.

According to a specific embodiment, Onivyde is administered prior to LV and 5-FU. Onivyde 70 mg/m² as an IV infusion over 90 minutes, followed by LV 400 mg/m² IV over 30 minutes, followed by 5-FU 2400 mg/m² IV over 46 hours, every 2 weeks. Patients homozygous for the UGT1A1*28 allele will initiate Onivyde® at 50 mg/m², and the dose can be increased if tolerated at later (e.g., 35 cycles).

The peptide is administered IV or SC with each irinotecan liposome dose every two weeks.

Efficacy testing can be done at any time, e.g., such as prior to, during or after the execution of the regimen as described herein e.g., following at least 1, 2, 3, 5 or more cycles of the therapy.

According to a specific embodiment, imagining is used for assessment of response. For example, CT or MRI may be used. The same imaging method may be preferably used throughout the treatment for each subject.

Alternatively or additionally, a biopsy can be performed using known tools available for those of skills in the art of pathology.

Alternatively or additionally, samples are collected for safety and efficacy analysis, e.g., CBC, anti-drug antibodies titer and determination of BL-8040 plasma concentrations.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It is expected that during the life of a patent maturing from this application many relevant chemotherapies for the treatment of pancreatic cancer will be developed and the scope of the term is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

In addition, any priority document(s) of this application is/are hereby incorporated herein by reference in its/their entirety.

1. A method of treating metastatic pancreatic adenocarcinoma in a subject in need thereof, the method comprising, administering to the subject a therapeutically effective amount of a peptide set forth in SEQ ID NO: 1 and a chemotherapy comprising irinotecan, fluorouracil (5-FU) and leucovorin (LV), thereby treating the metastatic pancreatic adenocarcinoma, wherein the subject is not subjected to treatment with an anti-PD-1.

2. (canceled)

3. The method of claim 1, wherein said anti PD-1 is pembrolizumab.

4. The method of claim 1, wherein said irinotecan is liposome-encapsulated.

5. The method of claim 1, wherein said irinotecan is Onivyde®.

6. The method of claim 1, wherein said peptide is administered subcutaneously (SC) or intravenously (IV).

7. The method of claim 1, wherein said peptide is administered at a dose of 1.25 mg/kg.

8. The method of claim 1, wherein said chemotherapy is administered intravenously.

9. The method of claim 1, wherein said irinotecan is administered prior to LV and 5-FU.

10. The method of claim 9, and wherein said irinotecan is administered at a dose of 70 mg/m² as an IV infusion over 90 minutes, followed by LV 400 mg/m² IV over 30 minutes, followed by 5-FU 2400 mg/m² IV over 46 hours, every 2 weeks.

11. The method of claim 1, wherein said peptide is administered with said irinotecan.

12. The method of claim 1, comprising an anti-histamine and optionally analgesics.

13. The method of claim 1, wherein said subject is post first-line treatment against said metastatic pancreatic adenocarcinoma.

14. The method of claim 13, wherein said first-line treatment comprises a gemcitabine-based chemotherapy.

15. The method of claim 1, wherein said metastatic pancreatic adenocarcinoma is unresectable.

16. The method of claim 1, wherein said metastatic pancreatic cancer is pancreatic ductal adenocarcinoma.

17. The method of claim 16, wherein said metastatic pancreatic ductal adenocarcinoma comprises intraductal papillary mucinous neoplasm.


SEQUENCE LISTING (1 sequence)

SEQ ID NO: 1 - protein, 14 aa, Artificial Sequence
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
XRXCYXKXPYRXCR